(19) United States
(12) Patent Application Publication
(10) Pub. No.: US 2023/0253109 A1
(43) Pub. Date: Aug. 10, 2023

(54) CANCER SCREENING DEVICE AND CANCER SCREENING METHOD

(71) Applicant: HITACHI, LTD., Tokyo (JP)

(72) Inventors: Takashi ISHIGAKI, Tokyo (JP); Minoru SAKAIRI, Tokyo (JP); Mayumi ABE, Tokyo (JP)

(21) Appl. No.: 18/011,211

(22) PCT Filed: Mar. 17, 2021
(86) PCT No.: PCT/JP2021/010804

§ 371 Date: Dec. 19, 2022

(30) Foreign Application Priority Data
Jul. 15, 2020 (JP) 2020-121399

(51) Int. Cl. G16H 50/20; G16B 20/00; G16B 40/20

(52) U.S. Cl. CPC G16H 50/20; G16B 20/00; G16B 40/20

(57) ABSTRACT

A cancer screening device includes a cancer screening model generation unit configured to construct, as a cancer screening model, clinical data that is results of cancer screening for a plurality of subjects including cancer patients and healthy subjects, and metabolite exhaustive data that is a result of LC/MS analysis of first urine specimens collected from these subjects and is information related to amounts of a plurality of metabolites in the first urine specimens, a second acquisition unit configured to acquire sample data that is a result of LC/MS analysis of a second urine specimen collected from a second subject who is different from the first subjects, and a screening processing unit configured to estimate the cancer state of the second subject by applying an amount of metabolite in the sample data to the cancer screening model, and an output processing unit configured to output the estimated state of the cancer.

### TECHNICAL FIELD

The present invention relates to a technology of a cancer screening device and a cancer screening method.

### BACKGROUND ART

In a conventional cancer screening system using urinary metabolites, biomarkers are narrowed down on the basis of, for example, information of two groups such as a cancer group and a non-cancer group, and a prediction value is calculated using a prediction formula according to the following formula (1). If the prediction value is a value of “+”, it is determined that a possibility of a cancer is high, and if the prediction value is “−”, it is determined that a possibility of a cancer is low. The prediction formula such as formula (1) is appropriately referred to as a cancer screening model.

The cancer screening model according to formula (1) is for identifying whether or not a cancer is present and is commonly used. The biomarkers are urinary metabolites having a causal relationship with onset of a cancer. In other words, although biomarkers are urinary metabolites, not all urinary metabolites are biomarkers. Hereinafter, the biomarkers will be referred to as markers, and the urinary metabolites will be referred to as metabolites.

Prediction value=α×(intensity of marker #1)+β×(intensity of marker #2)+γ×(intensity of marker #3)+δ (1)

α, β, γ, and δ in formula (1) are constants. Here, while formula (1) is a cancer screening model for determining whether or not a cancer is present, as described later, a prediction formula for determining whether or not a predetermined cancer type has developed and a state of a cancer will be also referred to as a cancer screening model.

Patent Literature 1 discloses a method for searching for biomarkers in urinary metabolites, including “searching for a urinary metabolite marker, including the steps of: (a) subjecting a urine specimen to a liquid chromatograph mass spectrometer (LC/MS) and analyzing a urinary metabolite in the urine specimen; (b) quantitatively evaluating an importance level of the urinary metabolite by a random forest method on the basis of analysis data of the urinary metabolite and selecting a urinary metabolite having a high importance level; (c) performing a discrimination analysis method using the analysis data of the selected urinary metabolite; and (d) determining a urinary metabolite associated with a specific disease or condition as a marker candidate on the basis of a result of the discrimination analysis”.

### CITATION LIST

### Patent Literature

Patent Literature 1: JP 2019-105456 A

### SUMMARY OF INVENTION

### Technical Problem

When a screening system using a cancer screening model according to formula (1) is put into practical use, the following problems arise.

(A) The cancer screening model according to formula (1) targets data whose correct answer (whether or not it is a cancer) is known, and the cancer screening model is created so as to have a high correct answer rate. Thus, if a prediction value by the prediction formula of formula (1) becomes even slightly positive, the system possibly determined that there is a possibility of a cancer. Conversely, if the prediction value by the prediction formula of formula (1) is even slightly negative, the system possibly determined that there is no possibility of a cancer. Although the cancer screening model based on the prediction formula of formula (1) is excellent, in the discriminant analysis, a magnitude of the calculated prediction value is not taken into account, and meaning is not given.

(B) When accuracy (sensitivity, specificity, AUC, etc.) of the constructed cancer screening model is verified, evaluation is performed using data for which whether or not there is a cancer is known. However, when this screening system is put into practical use, data for which an answer is unknown is often targeted, but the data is discriminated between two groups of whether or not there is a cancer. On the other hand, in practical use, it is desirable to indicate, as pre-screening, determination of a risk of developing a cancer, whether or not a cancer is in an early stage, or as a prognostic test, whether or not cancers are increased or decreased by treatment. For example, it is desired to obtain screening results other than two groups of whether or not there is a cancer, such as a size of a cancer, a degree of invasion, and the like. The technique described in PTL 1 also needs further improvement from such a viewpoint.

The present invention has been made in view of such a background, and an object of the present invention is to implement a variety of kinds of cancer screening.

### Solution to Problem

In order to solve the above-described problems, a cancer screening device of the present invention includes a first acquisition unit configured to acquire cancer screening data storing cancer screening results that are results of cancer screening for first subjects who are a plurality of subjects including cancer patients and healthy subjects and acquire first metabolite exhaustive data that is a result of analysis performed by LC/MS on first urine specimens collected from the first subjects and is information on amounts of a plurality of metabolites in the first urine specimens; a cancer screening model generation unit configured to construct, as a cancer screening model, a relationship between the cancer screening results in the cancer screening data and the respective amounts of the metabolites in the first metabolite exhaustive data on the basis of the cancer screening data and the first metabolite exhaustive data; and a second acquisition unit configured to acquire second metabolite exhaustive data that is a result of analysis performed by the LC/MS on a second urine specimen collected from a second subject who is a subject different from the first subjects, a cancer state estimation unit configured to estimate a state of a cancer in the second subject by applying an amount of the metabolites in the second metabolite exhaustive data to the cancer screening model, and an output unit configured to output the estimated state of the cancer.

Other solutions will be appropriately described in the embodiment.

### Advantageous Effects of Invention

According to the present invention, it is possible to implement a variety of kinds of cancer screening.

### BRIEF DESCRIPTION OF DRAWINGS

FIG. 1 is a view illustrating a configuration example of a cancer screening system according to the present embodiment.

FIG. 2 is a view illustrating a configuration example of a cancer screening device according to the present embodiment.

FIG. 3 is an example of a flowchart illustrating procedure of cancer screening model generation processing according to the present embodiment.

FIG. 4 is a view illustrating an example of clinical data.

FIG. 5 is a view illustrating an example of metabolite exhaustive data.

FIG. 6 is an example illustrating an example of marker candidate extraction result data.

FIG. 7 is a view illustrating a verification result obtained by applying test data to a first cancer screening model.

FIG. 8 is a view illustrating a verification result obtained by applying test data to a second cancer screening model.

FIG. 9 is a view illustrating a verification result obtained by applying test data to a third cancer screening model.

FIG. 10 is a view illustrating an example of cancer screening model data.

FIG. 11 is an example of a flowchart illustrating procedure of screening processing in the present embodiment.

FIG. 12 is a view illustrating an example of screening result data.

FIG. 13 is a view illustrating an example of result output data.

### DESCRIPTION OF EMBODIMENTS

Next, modes for carrying out the present invention (referred to as “embodiments”) will be described in detail with reference to the drawings as appropriate. Note that while the present embodiment is directed to colorectal cancer screening, the present invention is also applicable to screening for other cancers and is also applicable to a plurality of cancer types and general cancers.

<Cancer Screening System 10>

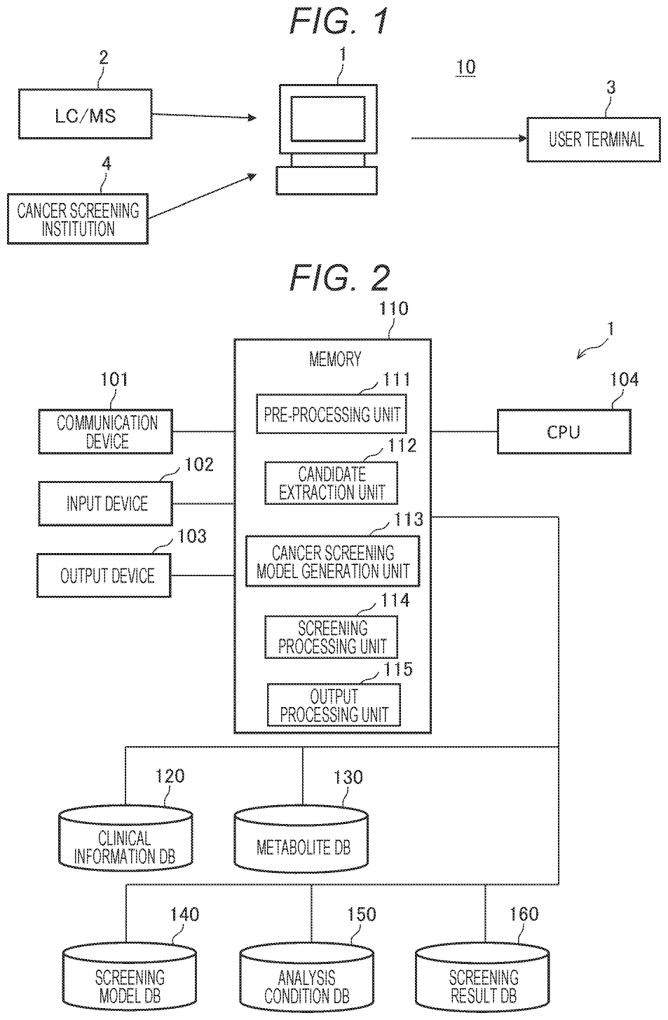

FIG. 1 is a view illustrating a configuration example of a cancer screening system 10 in the present embodiment.

The cancer screening system 10 includes a cancer screening device 1, a liquid chromatography-mass spectrometry (LC/MS) 2, and a user terminal (output unit) 3.

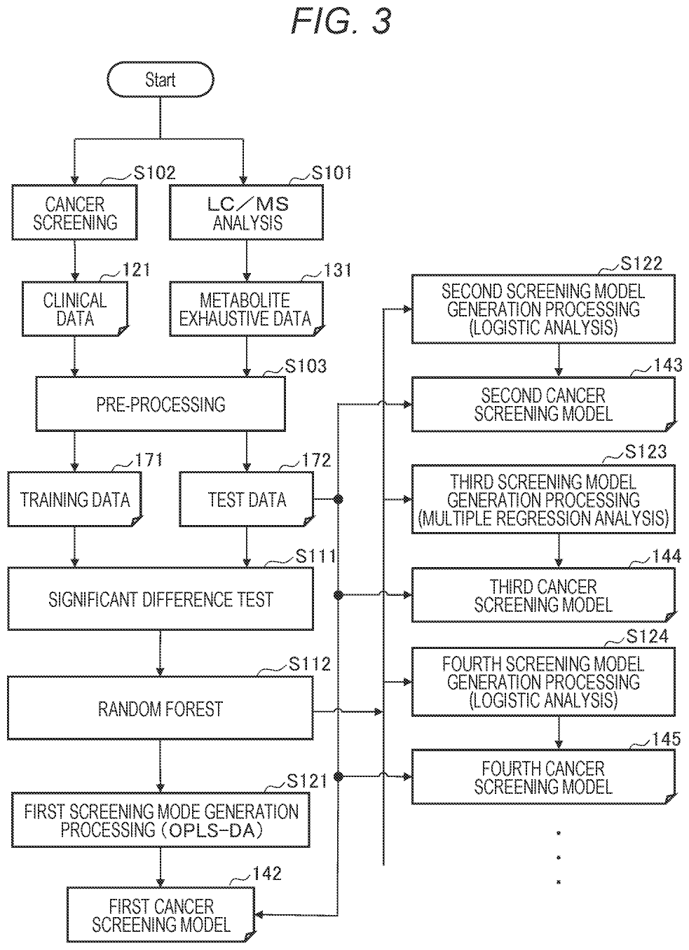

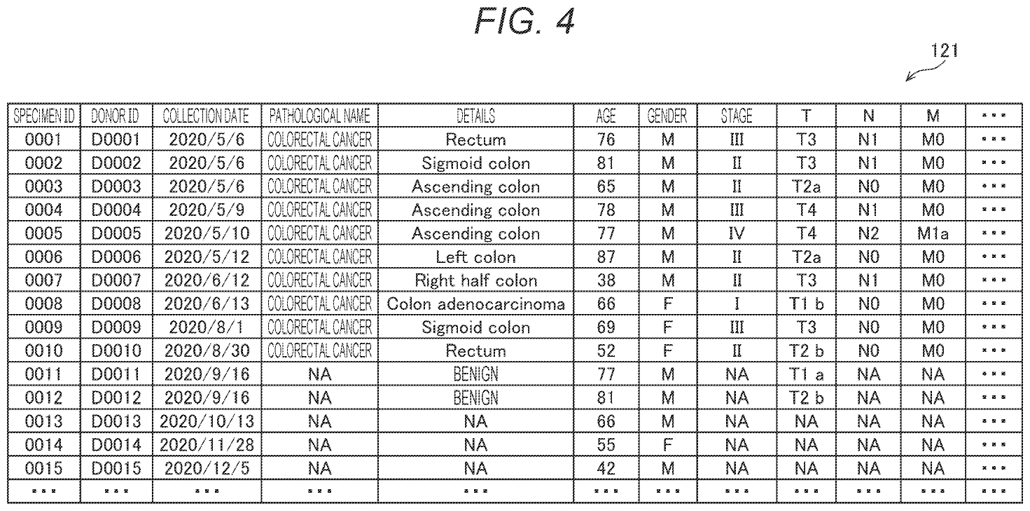

The cancer screening device 1 generates a cancer screening model on the basis of metabolite exhaustive data 131 (see FIG. 3) sent from the LC/MS 2 and clinical data 121 (see FIG. 3) sent from a cancer screening institution 4 (not illustrated). Here, the clinical data 121 is data of a result of cancer screening for a subject, and the metabolite exhaustive data 131 is data of a result of comprehensively detecting metabolites in a urine specimen using a plurality of separation modes by the LC/MS 2.

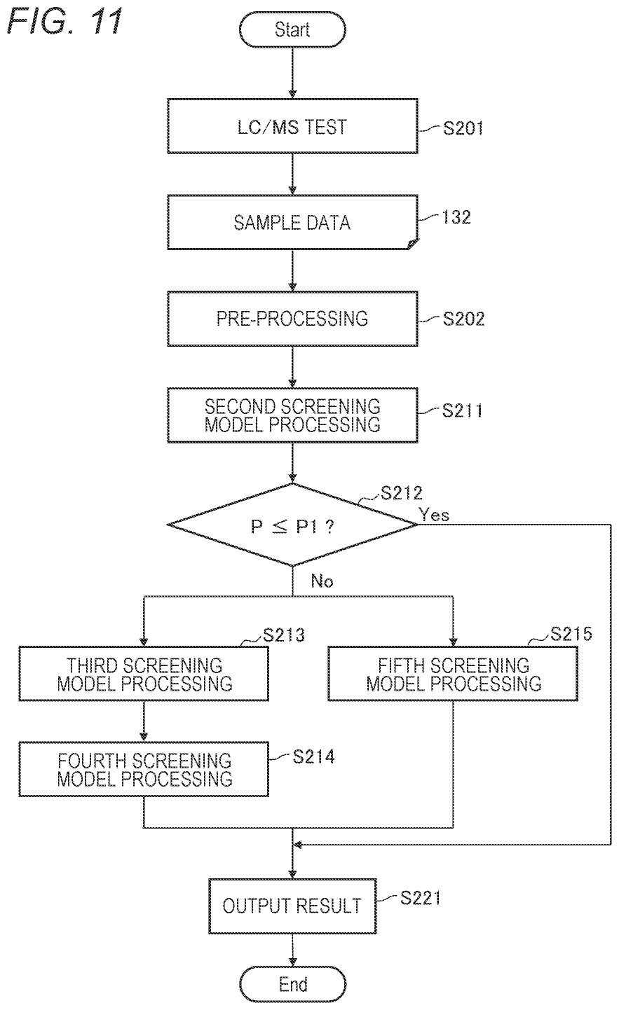

Furthermore, a urine specimen of a subject for whom cancer screening is desired to be performed is analyzed by the LC/MS 2, and sample data 132 (see FIG. 11), which is data on metabolites in the urine specimen, is input to the cancer screening device 1. Then, the cancer screening device 1 performs cancer screening on the basis of the sample data 132 and the generated cancer screening model. Then, the cancer screening device 1 outputs a result of the cancer screening to the user terminal 3 possessed by the subject.

Note that, in the present embodiment, generation of a cancer screening model and cancer screening using the generated cancer screening model are performed by one device. However, the present invention is not limited thereto, and generation of the cancer screening model and the cancer screening using the generated cancer screening model may be performed by different devices. Furthermore, in the example illustrated in FIG. 1, a result of the cancer screening using the cancer screening model is transmitted to the user terminal 3. However, the present invention is not limited thereto, and a paper, or the like, on which the result of the cancer screening is printed may be sent to the subject by mail.

<Cancer Screening Device 1>

FIG. 2 is a view illustrating a configuration example of the cancer screening device 1 according to the present embodiment. FIG. 1 will be referred to as appropriate.

The cancer screening device 1 includes a communication device (a first acquisition unit, a second acquisition unit) 101, an input device 102 such as a keyboard and a mouse, and an output device (an output unit) 103 such as a display and a printer. Further, the cancer screening device 1 also includes a memory 110 and a central processing unit (CPU) 104. Still further, the cancer screening device 1 includes a clinical information DB 120, a metabolite DB 130, a screening model DB 140, an analysis condition DB 150, and a screening result DB 160.

The communication device 101 transmits and receives information between the LC/MS 2, a server (not illustrated) provided in the cancer screening institution 4, and the user terminal 3.

A program stored in a storage device (not illustrated) is loaded into the memory 110. As a result of the loaded program being executed by the CPU 104, a pre-processing unit 111, a candidate extraction unit (narrowing unit) 112, a cancer screening model generation unit 113, a screening processing unit (cancer state estimation unit) 114, and an output processing unit (output unit) 115 are embodied.

The pre-processing unit 111 performs pre-processing on clinical data (cancer screening data) 121 (see FIG. 3) and metabolite exhaustive data (first metabolite exhaustive data) 131 (see FIG. 3). The pre-processing will be described later. The candidate extraction unit 112 extracts (narrows down) metabolites considered to be useful for cancer screening from metabolites in the metabolite exhaustive data 131.

The cancer screening model generation unit 113 generates a cancer screening model for determining various symptoms of cancers on the basis of metabolite data extracted by the candidate extraction unit 112 in the clinical data 121 and the metabolite exhaustive data 131.

The screening processing unit 114 performs cancer screening by using the generated cancer screening model and sample data (second metabolite exhaustive data) 132 (see FIG. 11). Here, the sample data 132 is a result of analysis of a urine specimen of a subject in which the subject is desired to take a cancer screening and the analysis is performed by the LC/MS 2, and is data related to an amount of metabolites contained in the urine specimen.

The output processing unit 115 transmits the result of the cancer screening to the user terminal 3, or the like.

The clinical information DB 120 stores clinical data 121 sent from the cancer screening institution 4. The clinical data 121 will be described later.

The metabolite DB 130 stores metabolite exhaustive data 131 which is a result of analysis performed by the LC/MS 2 and the sample data 132. The metabolite exhaustive data 131 and the sample data 132 will be described later.

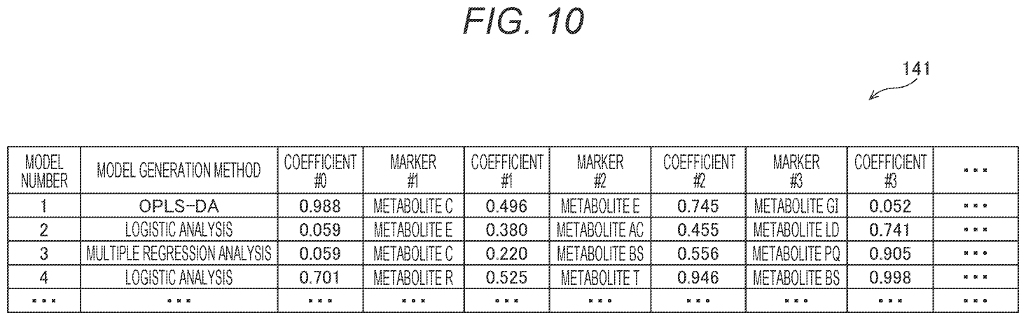

In the screening model DB 140, information regarding the cancer screening model generated by the cancer screening model generation unit 113 is stored as cancer screening model data 141 (see FIG. 10).

The analysis condition DB 150 stores conditions necessary for analysis performed by the LC/MS 2.

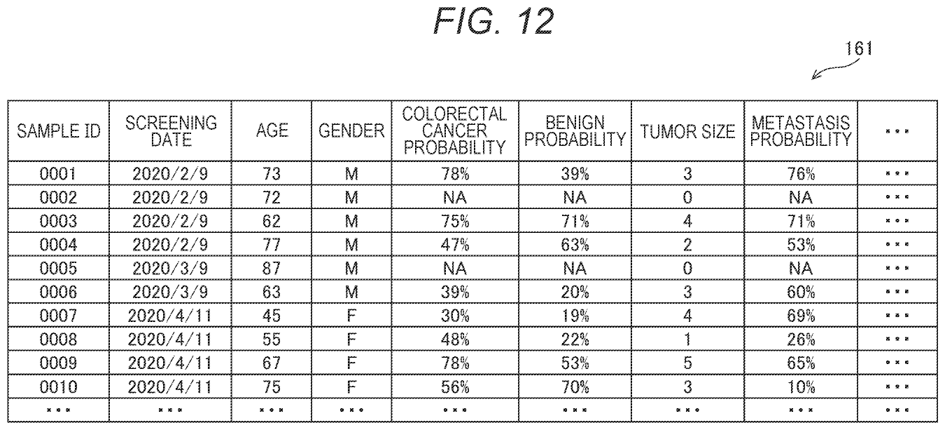

In the screening result DB 160, results of cancer screening performed by using the generated cancer screening model are stored as screening result data 161 (see FIG. 12).

As described above, the cancer screening device 1 performs two kinds of processing of processing of generating a cancer screening model and processing of performing actual cancer screening by using the generated cancer screening model. Hereinafter, the two kinds of processing will be described.

<Cancer Screening Model Generation Flowchart>

FIG. 3 is an example of a flowchart illustrating procedure of cancer screening model generation processing in the present embodiment.

First, analysis (LC/MS analysis) by the LC/MS 2 is performed on a urine specimen collected from a subject (S101), and cancer screening is performed for the subject (S102). In step S101, metabolites in the urine specimen are comprehensively detected by using a plurality of separation modes. Here, as a plurality of separation modes, in order to detect as many metabolites in the urine specimen as possible, separation in LC such as reversed phase, normal phase, HILIC, or the like, positive or negative ionization in MS by using an electrospray method, or the like, is used. The result of the cancer screening is stored in the clinical data 121, and the result of the analysis by the LC/MS 2 is stored in the metabolite exhaustive data 131.

Then, the clinical data 121 and the metabolite exhaustive data 131 are input to the cancer screening device 1.

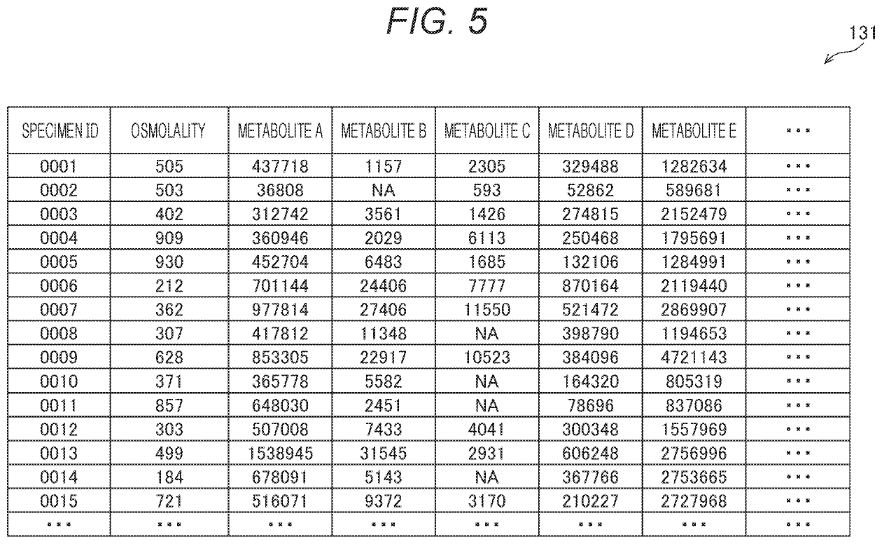

Here, specific examples of the clinical data 121 and the metabolite exhaustive data 131 will be described with reference to FIGS. 4 and 5.

In the present embodiment, 30 urine specimens are collected from colorectal cancer patients and healthy subjects as a control group and analyzed by the LC/MS 2 as described above (S101). As a result, 1000 or more intensities of ions of the metabolites can be obtained.

(Clinical Data 121)

FIG. 4 is a view illustrating an example of the clinical data 121.

The clinical data 121 includes fields of “specimen ID”, “donor ID”, “collection date”, “pathological name”, “details”, “age”, “gender”, “stage”, “T factor (T)”, “N factor (N)”, and “M factor (M)”.

Here, the “specimen ID” is an ID for uniquely distinguishing a urine specimen.

The “donor ID” is an ID for uniquely distinguishing a subject (donor).

The collection date is date on which the urine specimen is collected.

“Pathological name” is a name of a cancer found as a result of cancer screening. Note that “NA” means that the cancer is benign or no cancer has been detected.

The “details” stores detailed information of the detected cancer (colorectal cancer). In the example of FIG. 4, information of places where colorectal cancer has been detected, such as “rectum” and “sigmoid colon”, are stored. As illustrated in the example of FIG. 4, when the cancer (tumor) is benign, “benign” is input, and when the cancer (tumor) itself is not detected, “NA” is input.

The “T factor (T)” is an index indicating a size of the tumor and a degree of invasion, and is T1a, T1b, T2a, T2b, T3, and T4 in the order of ascending symptoms from the mildest symptom.

“N factor (N)” is an index indicating a degree of lymph node metastasis of a tumor, and “NO” indicates a case where there is no lymph node metastasis, and “N3” indicates a case where there is the most metastasis.

“M factor (M)” is an index indicating distant metastasis, “M1a” or “M1b” indicates a case where there is distant metastasis and a location of metastasis, and “M0” indicates a case where there is no distant metastasis.

As illustrated in FIG. 4, it is desirable to collect detailed clinical information as much as possible. In addition, as illustrated in FIG. 4, it is desirable to collect a lot of detailed information such as a size and a stage of the tumor, malignancy/benignancy, TNM, and the like, in addition to whether or not the tumor is a cancer.

(Metabolite Exhaustive Data 131)

FIG. 5 is a view illustrating an example of the metabolite exhaustive data 131.

As illustrated in FIG. 5, the metabolite exhaustive data 131 includes fields of “specimen ID”, “osmolality”, “metabolite A”, “metabolite B”, “metabolite C”, . . . .

The “specimen ID” is the same as the “specimen ID” in FIG. 4.

Each field of “metabolite A”, “metabolite B”, “metabolite C”, . . . stores information of ion intensity (hereinafter, referred to as intensity) of each metabolite in a urine specimen measured by the MS. Metabolites can be discriminated by a metabolite database (not illustrated), or the like, and also include unknown metabolites with an unknown chemical structure because only an m/z (mass-to-charge ratio) is known at the time of MS. In addition, as illustrated in FIG. 5, it is desirable to correct amounts of metabolites to amounts of metabolites per day by measuring the osmolality (or creatinine concentration) of each urine specimen.

The explanation is back to FIG. 3.

Next, in step S103, the pre-processing unit 111 performs pre-processing to the input clinical data 121 and metabolite exhaustive data 131. The pre-processing unit 111 performs data association, data integration, unnecessary data cleaning, format conversion, normalization, normalization by osmolality or creatinine concentration, standardization, missing value complementation, outlier exclusion, autoscaling, and the like, as necessary. In this process, drugs that are not included in the cancer screening model, exogenous metabolites derived from foods, and the like, are also excluded. Note that it is not necessary to perform all the pre-processing described here. Note that the processing in step S103 may be performed by the pre-processing unit 111 on the basis of information input by the user via the input device 102 on the basis of experience or may be automatically performed by the pre-processing unit 111.

Subsequently, the pre-processing unit 111 divides the urine specimen data in each of the pre-processed clinical data 121 and metabolite exhaustive data 131 into training data 171 for generating a cancer screening model and test data 172 for verifying the generated cancer screening model, as necessary. Here, the urine specimen data is a record having a specimen ID common to the clinical data 121 and the metabolite exhaustive data 131. The urine specimen data is randomly divided into the training data 171 and the test data 172. Note that the training data 171 is teacher data for generating a cancer screening model. The test data 172 is data for verifying the generated model. As verification, cross verification is performed.

Next, the candidate extraction unit 112 performs marker candidate extraction processing. Here, the candidate extraction unit 112 first performs a significance test (t-test, f-test, Wilcoxon rank sum test, etc.) on an amount of each metabolite in a urine specimen for two groups of cancer patients and healthy persons (S111). Then, the candidate extraction unit 112 extracts metabolites having a significant difference between cancer patients and healthy subjects as marker candidates. Further, the candidate extraction unit 112 performs correlation analysis and a random forest method which is one of machine learning (S112), calculates importance levels of the marker candidates and ranks the marker candidates. The processing in steps S111 and S112 may be executed at the time of generating each cancer screening model (S121 to S124). However, the number of metabolite types in the metabolite exhaustive data 131 is as large as several thousands, and thus, by narrowing down the number of marker candidates from several tens to several hundred in step S111 in advance, a calculation amount and a calculation period are reduced. Note that both the significance test (S111) and the random forest method (S112) do not need to be performed, and either one may be performed.

(Marker Candidate Extraction Result)

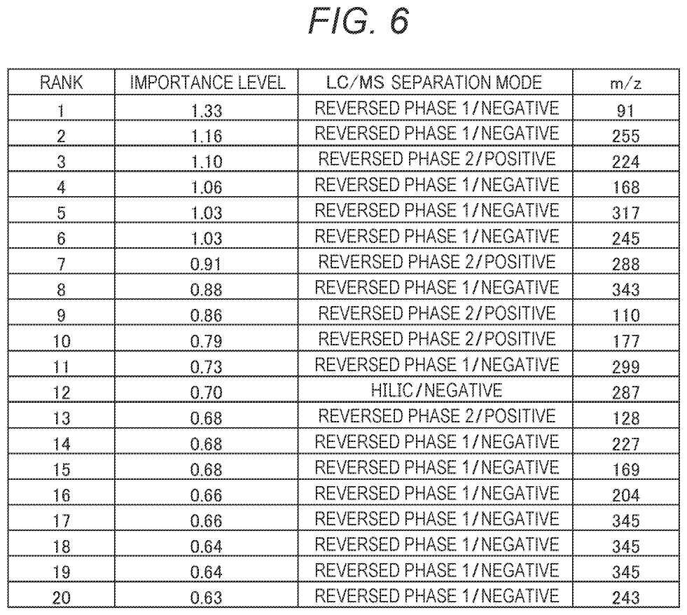

As a result of the processing in steps S111 and S112, the top 20 marker candidates obtained are illustrated in FIG. 6. FIG. 6 is an example illustrating an example of marker candidate extraction result data.

The marker candidate extraction result data includes fields of “rank”, “importance level”, “LS/MS separation mode”, and “m/z (mass-to-charge ratio)”.

Here, the “importance level” is a degree of importance calculated by random forest. In addition, in the example of FIG. 6, “ranks” are given in descending order of “importance level”. In addition, in the example of FIG. 6, while the same separation mode and the same mass are set in the ranks 17, 18 and 19, the ranks 17, 18 and 19 are distinguished by a difference in retention time in LC, and the like. In addition, there are compounds having the same chemical formula and the same mass but having different chemical structures such as optical isomers.

The explanation is back to FIG. 3.

Next, the cancer screening model generation unit 113 performs first screening model generation processing (S121). In step S121, the cancer screening model generation unit 113 uses OPLS-DA (orthogonal partial least squares discriminant analysis) to generate a first cancer screening model (first cancer screening model) 142 that is a cancer screening model for determining whether or not it is a cancer. In the present embodiment, data of colorectal cancer patients/healthy subjects are handled, and thus, whether or not it is a colorectal cancer is determined by the first cancer screening model 142. Note that not only OPLS-DA but also other discrimination analysis may be used.

For example, the cancer screening model generation unit 113 selects top 20 marker candidates among 10 marker candidates indicated in the marker candidate extraction result illustrated in FIG. 6 as markers. In addition, 60 pieces of urine specimen data are divided into 30 pieces of training data 171 and 30 pieces of test data 172. As described above, the urine specimen data is a record having a specimen ID common to the clinical data 121 and the metabolite exhaustive data 131. Note that “10” and “30” of the top 10 markers, 30 pieces of training data 171, and 30 pieces of test data 172 are examples, and the number is not limited to these. For example, a combination of a record of “specimen ID: 0001” in the clinical data 121 of FIG. 4 and a record of “specimen ID: 0001” in the metabolite exhaustive data 131 of FIG. 5 will be referred to as one piece of urine specimen data. In addition, 60 pieces of urine specimen data are randomly selected, and 30 pieces of training data 171 and 30 pieces of test data 172 are different urine specimen data.

Then, the cancer screening model generation unit 113 first generates the first cancer screening model 142 for discriminating between a colorectal cancer (cancer) and healthy subjects by OPLS-DA using the training data 171. In other words, the first cancer screening model 142 determines whether or not a colorectal cancer has developed.

Specifically, the cancer screening model generation unit 113 temporarily sets a linear expression having 10 variables as intensity of 10 markers. Next, the cancer screening model generation unit 113 uses OPLS-DA to calculate a coefficient of each variable that can be discriminated between colorectal cancer patients and healthy subjects. As a result, the first cancer screening model 142 represented by the following formula (2) is generated.

y0=a1·x1+a2x2+ . . . +a9x9+a10x10+a0 (2)

Here, x1, x2, . . . , and x10 are intensity of the selected top 10 markers among the 20 marker candidates indicated in the marker candidate extraction result illustrated in FIG. 6. In addition, a1, a2, . . . , a10, and a0 are coefficients of each variable that discriminate between colorectal cancer patients/healthy subjects, calculated using OPLS-DA.

Thereafter, the cancer screening model generation unit 113 verifies the generated first cancer screening model 142 using 30 pieces of test data 172. In other words, the cancer screening model generation unit 113 applies the first cancer screening model 142 to the data with answers of colorectal cancers/healthy subjects and verifies a correct answer rate. Note that step S121 includes generation of the first cancer screening model 142 by using the OPLS-DA to verification of the generated first cancer screening model 142.

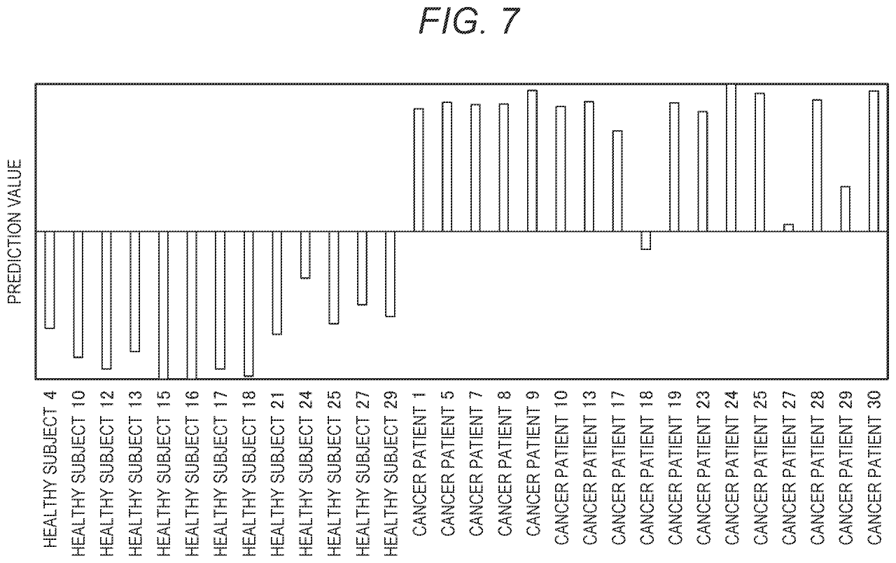

FIG. 7 is a view illustrating a verification result obtained by applying the test data 172 to the first cancer screening model 142.

A prediction value y0 generated by using the first cancer screening model 142 indicated in formula (2) is a first-order polynomial obtained by multiplying intensity of 10 markers by a coefficient for each marker and the cancer screening model identifies a cancer patient when the prediction value y0 is positive and identifies a healthy subjects when the prediction value y0 is negative.

According to verification using 30 pieces of test data 172 illustrated in FIG. 7, all 15 healthy subjects known to be healthy subjects in advance were determined as healthy subjects. In addition, it shows that a discriminant model with relatively high accuracy was generated.

The explanation is back to FIG. 3.

After step S112 in FIG. 3, the cancer screening model generation unit 113 performs second screening model generation processing (S122). In step S122, the cancer screening model generation unit 113 generates a second cancer screening model (second cancer screening model) 143 by using logistic analysis. Here, the second cancer screening model 143 is a cancer screening model that calculates a probability (risk) of onset of a cancer or a predetermined cancer type. In the present embodiment, data of colorectal cancer patients/healthy subjects is used, and thus, a probability of onset of a cancer (colorectal cancer in this case) is determined with the second cancer screening model 143.

It is supposed that the second cancer screening model 143 is a scheme, for example, in which a subject collects urine at home, a risk is easily assessed and a thorough examination is suggested. First, the cancer screening model generation unit 113 divides 60 pieces of urine specimen data used in the first cancer screening model 142 into 30 pieces of training data 171 and test data 172, the same as the first cancer screening model 142 and performs logistic analysis on the training data 171. In this process, the cancer screening model generation unit 113 temporarily sets the following formula (3).

y1=1/[1+exp{−(b1·x1+b2x2+ . . . +b20·x20+b0)}] (3)

Here, x1, x2, . . . , x20 are intensity of 20 marker candidates used in the first cancer screening model 142. A suffix of x is “rank” indicated in FIG. 6. In addition, b1, b2, . . . , and b20 are coefficients and are determined by a maximum likelihood method.

Next, the cancer screening model generation unit 113 calculates an odds ratio (exp(b1), exp(b2), . . . , exp(b20)) for each marker candidate. Thereafter, the cancer screening model generation unit 113 selects top 7 markers in descending order of the odds ratio. The number of markers to be selected is not limited to seven. As a result, seven markers in positions 1, 2, 5, 7, 11, 12, and 20 are selected in the “rank” illustrated in FIG. 6.

Then, when the cancer screening model generation unit 113 applies the selected 7 markers to formula (3), the second cancer screening model 143 of the following formula (4) is obtained. Here, the cancer screening model is reconstructed with seven markers, and thus, each of the coefficients b1, b2, b5, b7, b11, b12, b20, and b0 has a value different from that in formula (3).

y1=1/[1+exp{−(b1·x1+b2x2+b5·x5+b7·x7+b11·x11+b12·x12+b20·x20+b0)}] (4)

x1, x2, . . . , b1, b2, . . . are the same as those in formula (3), and thus, description thereof is omitted here. A portion of y2 in exp{-(y2)} in formula (4), that is, the following formula (5) is set as a prediction value of the second cancer screening model 143.

y2=b1·x1+b2·x2+b5·x5+b7·x7+b11·x11+b12·x12+b20·x20+b0 (5)

Thereafter, the cancer screening model generation unit 113 verifies the second cancer screening model 143. Specifically, the cancer screening model generation unit 113 substitutes intensity of the selected marker in the 30 pieces of test data 172 into the second cancer screening model 143 (formula (4)). Then, the cancer screening model generation unit 113 compares the probability (formula (4)) obtained from the probability with onset of a cancer in the test data 172, thereby verifying the second cancer screening model 143. Step S122 includes steps from generation of the second cancer screening model 143 by logistic analysis to verification of the generated second cancer screening model 143.

When logistic analysis was performed by using actual urine specimen data to obtain an odds ratio, it was found that the rank by the odds ratio did not always coincide with the rank by the random forest indicated in FIG. 6, as shown in this example. In other words, by using the second cancer screening model 143 in addition to the random forest, highly accurate cancer (colorectal cancer) screening may be implemented.

In general, a degree of matching of ranks between different analysis methods, such as the rank in the random forest and the rank obtained through the logistic analysis as described above, is improved as the number of markers to be used is larger. In addition, as the number of markers to be used is larger, a cancer screening model with higher accuracy may be generated. On the other hand, as the number of markers to be used is smaller, time and cost for cancer screening using a cancer screening model tend to be decreased. Thus, the number of markers to be used is determined by the user on the basis of these balances.

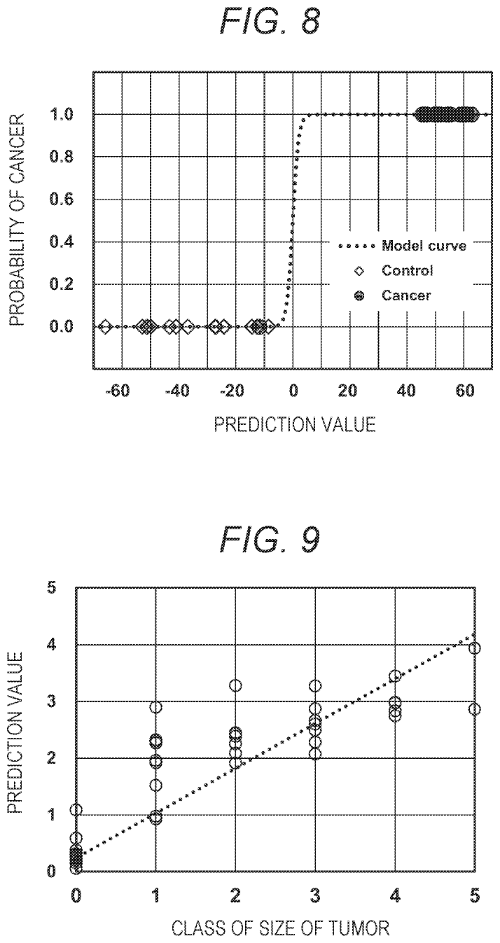

FIG. 8 is a view illustrating a verification result obtained by applying the test data 172 to the second cancer screening model 143.

In FIG. 8, a horizontal axis represents a prediction value that is a first-order polynomial obtained by multiplying the intensity of the seven target markers by respective coefficients.

Here, the prediction value is a value of y2 in formula (5).

Then, a vertical axis is a probability of onset of a cancer (colorectal cancer) with respect to the prediction value y2 of the second cancer screening model 143 (y1 in formula (4)).

In FIG. 8, a dotted line indicates a probability (y1 in formula (4)) of onset of a cancer (colorectal cancer) with respect to the prediction value y2 obtained by using the second cancer screening model 143. Furthermore, outlined diamonds and dot circles indicate the prediction value y2 in a case of using 7 markers in the test data 172, and a probability of onset of a cancer (colorectal cancer) obtained by using the second cancer screening model 143 (y1 in formula (4)). The outlined diamonds are results based on data of urine specimen collected from subjects known as healthy subjects, and the dot circles are results based on urine specimens collected from subjects known as cancer patients. In the example of FIG. 8, almost all the probabilities based on the prediction values of the test data 172 are divided into “0” and “1”, but actually, there are probabilities between “0” and “1”.

As indicated in FIG. 8, the second cancer screening model 143 calculates the probability of onset of a colorectal cancer with high accuracy. The second cancer screening model 143 outputs a qualitative probability for a colorectal cancer patient/healthy subject. As described above, by applying the second cancer screening model 143 to a urine specimen collected from a subject who is unknown to have a colorectal cancer or not, it is possible to score (risk) the probability of onset of a colorectal cancer. This makes it possible to present a risk of a colorectal cancer to the subject.

The explanation is back to FIG. 3.

After step S122 in FIG. 3, the cancer screening model generation unit 113 performs third screening model generation processing (S123). In step S123, the cancer screening model generation unit 113 generates a third cancer screening model (third cancer screening model) 144 by using multiple regression analysis. The third cancer screening model 144 is a cancer screening model for estimating a size of a tumor.

Here, the user first divides a size of the tumor into five classes from “1” to “5” and sets a class of “0” for a person without tumor. Next, the cancer screening model generation unit 113 performs multiple regression analysis on the training data 171 used at the time of generating the first cancer screening model 142. Specifically, the cancer screening model generation unit 113 temporarily sets the following formula (11).

y4=c1·x1+c2·x2+ . . . +c20·x20+c0 (11)

Here, x1, x2, . . . , x20 are intensity of 20 marker candidates used in the first cancer screening model 142. A suffix of x is “rank” indicated in FIG. 6. Further, c1, c2, . . . , c20, and c0 are coefficients (partial regression coefficients). These coefficients are determined by a general multiple regression analysis method. Then, the cancer screening model generation unit 113 selects, from the 20 markers, six markers with the “ranks” of 1, 2, 5, 8, 9, and 10 illustrated in FIG. 6. This selection is determined in consideration of a t value and a p value of each marker in the multiple regression analysis and accuracy of the third cancer screening model 144 in the cross validation. In addition, y4 represents a size of the tumor from “0” to “5”.

Then, the cancer screening model generation unit 113 sets, as a third cancer screening model 144, the following formula (12) obtained by applying the selected six markers with the “ranks” of 1, 2, 5, 8, 9, and 10 illustrated in FIG. 6 to formula (11). Here, the coefficients c1, c2, c5, c8, c9, c10, and c0 are values different from those in formula (11) because the cancer screening model is reconstructed with these six markers.

y4=c1·x1+c2·x2+c5·x5+c8·x8+c9·x9+c10·x10+c0 (12)

Next, the cancer screening model generation unit 113 verifies the third cancer screening model 144. Specifically, the cancer screening model generation unit 113 substitutes the intensity of metabolites in the test data 172 into the third cancer screening model 144 indicated in formula (12). Then, the cancer screening model generation unit 113 compares the result obtained by substituting the intensity of metabolites in the test data 172 into the third cancer screening model 144 with the size of the tumor in the test data 172, thereby verifying the third cancer screening model 144. Note that the processing in step S123 includes generation and verification of the third cancer screening model 144 by multiple regression analysis.

FIG. 9 is a view illustrating a verification result obtained by applying the test data 172 to the third cancer screening model 144.

In FIG. 9, a horizontal axis represents a class of a size of the tumor (“0” to “5”). In addition, a vertical axis represents a prediction value (y4 in formula (12)) by the third cancer screening model 144. Then, white circles in FIG. 9 indicate values obtained by applying the test data 172 to the third cancer screening model 144. For example, white circles plotted on “1” on the horizontal axis indicate results of applying the third cancer screening model 144 to urine specimen data whose tumor size is known as according to a result of cancer screening (colorectal cancer screening in the present embodiment).

As indicated in FIG. 9, the third cancer screening model 144 can roughly estimate the size of the tumor. A cancer screening model for estimating a therapeutic effect, or the like, can be generated by processing similar to that of the third cancer screening model 144.

Note that the prediction value on the horizontal axis in FIG. 8 is a prediction value by the second cancer screening model 143 (formula (4)), and the prediction value on the vertical axis in FIG. 9 is a prediction value by formula (12) (third cancer screening model 144). In other words, the prediction value on the vertical axis in FIG. 9 is a value obtained by substituting concentration of the metabolites in the test data 172 into formula (12) (y4 in formula (12)).

The explanation is back to FIG. 3.

After step S123 in FIG. 3, the cancer screening model generation unit 113 performs fourth screening model generation processing (S124). In step S124, the cancer screening model generation unit 113 generates a fourth cancer screening model (second cancer screening model) 145 by using logistic analysis. The fourth cancer screening model 145 is a cancer screening model for calculating a probability (risk; malignant/benign probability of a tumor) that a tumor of a cancer or a predetermined cancer type (in the present embodiment, colorectal cancer) is malignant or benign.

The generation procedure of the fourth cancer screening model 145 is the same procedure as the generation of the second cancer screening model 143, and thus, description thereof is omitted here. In the present embodiment, the fourth cancer screening model 145 estimates a malignant/benignancy probability of a tumor of a colorectal cancer, but the present invention is not limited thereto, and a probability of metastasis to other sites of the colorectal cancer may be used, or other qualitative probabilities may be estimated.

The generated respective cancer screening models are stored in the cancer screening model data 141 illustrated in FIG. 10 and then stored in the screening model DB 140.

Here, whether or not a cancer has developed is estimated by the first cancer screening model 142, a probability of onset of a cancer (colorectal cancer in the present embodiment) is estimated by the second cancer screening model 143, a size of a tumor is estimated by the third cancer screening model 144, and malignant/benign probability of a tumor of cancer (colorectal cancer in the present embodiment) is estimated by the fourth cancer screening model 145. In addition, by performing multiple regression analysis as in the third cancer screening model 144, it is possible to generate a cancer screening model for estimating a therapeutic effect, a degree of invasion of cancer, and the like.

(Cancer Screening Model Data 141)

FIG. 10 is a view illustrating an example of cancer screening model data 141.

The cancer screening model data 141 illustrated in FIG. 10 is generated in the cancer screening model generation processing (S121 to S124) illustrated in FIG. 3.

As illustrated in FIG. 10, the cancer screening model data 141 includes fields of “model number”, “model generation method”, “coefficient #0”, “marker #1”, “coefficient #1”, “marker #2”, “coefficient #2”, “marker #3”, “coefficient #3”, . . . .

In the “model number”, the number of the cancer screening model is stored. For example, “model number: 1” indicates the first cancer screening model 142 described above, and “model number: 2” indicates the second cancer screening model 143 described above. The same applies to “model number: 3” and “model number: 4”. Note that information indicating what is to be estimated by using each cancer screening model is also preferably stored in the cancer screening model data 141. For example, the third cancer screening model 144 is a model for estimating “a size of a tumor”.

In the “model generation method”, the name (OPLS-DA, logistic analysis, multiple regression analysis, and the like) of the analysis method used when each cancer screening model is generated is stored.

In the “coefficient #0”, a value of a zero-order coefficient in each cancer screening model is stored. The zero-order coefficient is b0 in formula (5) and c0 in formula (12).

“Marker #1” is x1 in formula (5) or (12), and “coefficient #1” is b1 in formula (5) or c1 in formula (12).

Hereinafter, the same applies to “marker #2”, “marker #3”, . . . , “coefficient #2”, “coefficient #3”, —. Note that the number after “#” is a number in the cancer screening model and is not the “rank” in FIG. 6. For example, “marker #3” in the second cancer screening model 143 is x5 in formula (5), and “coefficient #3” is b5 in formula (5). Similarly, “marker #3” in the third cancer screening model 144 is x5 in formula (12), and “coefficient #3” is c5 in formula (12).

As qualitative variables determined by the cancer screening model, in addition to those described in the present embodiment, presence or absence of cancer metastasis, presence or absence of invasion, presence or absence of angiogenesis, presence or absence of metabolic reprogramming (reflection on metabolites), and the like, are also possible. In addition, as quantitative variables determined by the cancer screening model, a degree of activity, a cancer stage, a degree of invasion, the number of angiogenesis, a degree of metabolic reprogramming, and the like, are also possible. In addition, a location of the cancer, the name of the disease, and the like, can also be determined by complexly determining the qualitative variable and the quantitative variable. These cancer screening models are generated as exhaustively as possible, and the most suitable one is used in cancer screening to be performed.

Note that there is a set of markers suitable for each cancer screening model. In each cancer screening model, the marker to be used is made common as much as possible, and the number of markers is reduced, so that efficiency of cancer screening in FIG. 11 may be improved.

### <Screening Flowchart>

FIG. 11 is an example of a flowchart illustrating procedure of screening processing in the present embodiment.

In FIG. 11, actual cancer screening is performed using the cancer screening model generated by the flowchart illustrated in FIG. 3. Here, performing colorectal cancer screening as cancer screening is exemplified.

First, analysis by the LC/MS 2 (LC/MS analysis) is performed on a urine specimen of a subject (S201), thereby measuring intensity of each metabolite.

Then, the sample data 132 is input to the cancer screening device 1. The sample data 132 may be similar to the metabolite exhaustive data 131 illustrated in FIG. 5, but it is desirable that osmolality or a creatinine amount is added to the metabolite exhaustive data 131 illustrated in FIG. 5. In the input sample data 132, the metabolite may be input for the metabolite used in each cancer screening model to be used.

Then, the pre-processing unit 111 performs pre-processing on the input sample data 132 (S202). The processing in step S202 is similar to that in step S104 in FIG. 3, and thus, description thereof is omitted here.

Subsequently, the screening processing unit 114 calculates a probability P of onset of a colorectal cancer for the urine specimen to be screened by using the second cancer screening model 143 (S211, second screening model processing). In other words, the screening processing unit 114 calculates the prediction value by substituting the intensity of the marker in the sample data 132 into formula (5). Further, the screening processing unit 114 calculates the probability P of onset (that is, y1 in formula (4)) of a colorectal cancer by substituting the calculated prediction value (y2 in formula (5)) into formula (4). Here, the second cancer screening model 143 is used, but presence or absence of onset of a cancer may be determined by using the first cancer screening model 142.

Then, the screening processing unit 114 determines whether or not the probability P of onset of a colorectal cancer calculated in the second screening model processing of step S211 is equal to or less than a predetermined value P1 (P≤P1) (S212). Here, P1=10%, but the probability is not limited thereto. Here P1=10% is set, but by setting P1=0%, the screening processing unit 114 may determine whether or not P=P1 is met in step S212.

As a result of step S212, when the probability P of onset of a colorectal cancer is equal to or less than the predetermined value P1 (here, 10%) (S212: Yes), the screening processing unit 114 outputs the result of the cancer screening (here, colorectal cancer screening) to the user terminal 3 as determination of a low risk (for example, “D” for the ABCD grading scale) of a colorectal cancer (S221).

As a result of step S212, when the probability P of onset of a colorectal cancer is higher than the predetermined value P1 (here, 10%) (S212: No), that is, when the probability of onset of a colorectal cancer is high or moderate in the cancer screening model, the screening processing unit 114 executes the next cancer screening model and calculates and outputs a more detailed state.

In the example of the flowchart illustrated in FIG. 11, if “No” is determined in step S212, the screening processing unit 114 performs third screening model processing (S213). In this processing, the screening processing unit 114 calculates a prediction value of the size of the tumor by using the third cancer screening model 144 (formula (12)) and stores the result in the screening result data 161.

Further, the screening processing unit 114 performs fourth screening model processing (S214). In this processing, the screening processing unit 114 calculates a malignant/benign probability of a tumor of a cancer (here, a colorectal cancer) by using the fourth cancer screening model 145 and stores the result in the screening result data 161 (see FIG. 12).

If “No” is determined in step S212, the screening processing unit 114 performs the fifth screening model processing (S215). In this processing, the probability of metastasis of a cancer to other parts is calculated by using the fifth cancer screening model, and the result is stored in the screening result data 161. As described above, although not illustrated in FIG. 3, the fifth cancer screening model is a cancer screening model that calculates presence or absence of cancer metastasis and the probability thereof.

In FIG. 11, the fourth screening model processing (S214) is performed after the third screening model processing (S213), and the fifth screening model processing (S215) is performed in parallel with the third screening model processing and the fourth screening model processing, but the processing order is not limited thereto. Furthermore, the example of FIG. 11 is an example, and what type of cancer screening model is used for performing cancer screening depends on setting by the user.

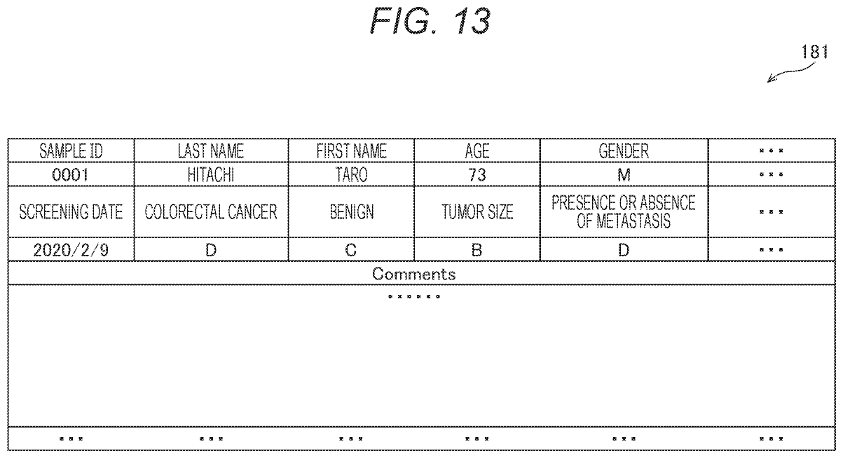

Finally, the output processing unit 115 generates result output data 181 illustrated in FIG. 13 on the basis of content of the screening result data 161. Then, the output processing unit 115 outputs the result of the cancer screening (here, colorectal cancer screening) by outputting the result output data 181 to the user terminal 3 (S221).

(Screening Result Data 161)

FIG. 12 is a view illustrating an example of the screening result data 161.

The screening result data 161 illustrated in FIG. 12 is generated in the screening processing illustrated in FIG. 11.

As illustrated in FIG. 12, the screening result data 161 has fields such as “sample ID”, “screening date”, “age”, “gender”, “colorectal cancer probability”, “benign probability”, “tumor size”, “metastasis probability”, . . . .

The “sample ID” is an ID for uniquely distinguishing a urine specimen. The urine specimen here is a urine specimen in the sample data 132.

The “colorectal cancer probability” is a probability of onset of a colorectal cancer calculated by the second cancer screening model 143.

In addition, the “benign probability” is a malignant/benign probability of a tumor of a colorectal cancer calculated by the fourth cancer screening model 145.

“Tumor size” is a class of a size of a tumor calculated by the third cancer screening model 144.

The “metastasis probability” is calculated by the fifth cancer screening model.

(Result Output Data 181)

FIG. 13 is a view illustrating an example of the result output data 181.

The result output data 181 illustrated in FIG. 13 is generated and output by the output processing unit 115 on the basis of the screening result data 161 illustrated in FIG. 12. The output is performed on a mobile terminal, or the like, of the subject.

As illustrated in FIG. 13, the result output data 181 is “sample ID”, “last name”, “first name”, “age”, “gender”, “screening date”, “colorectal cancer probability (colorectal cancer)”, “benign probability (benign)”, “tumor size”, and “metastasis probability (presence or absence of metastasis)”. For “last name” and “first name”, there is name data (not illustrated) in which “sample ID” and the subject's last and first name are associated, and the output processing unit 115 searches for the name data using “sample ID” as a key, so that “last name” and “first name” are output to the result output data 181.

The data stored in the result output data 181 is data stored in the screening result data 161 of FIG. 12. In the example illustrated in FIG. 13, data stored in a record of “sample ID: 0001” of the screening result data 161 in FIG. 12 is illustrated. However, the “colorectal cancer probability” is indicated in four stages of “A” to “D” in ascending order of the “colorectal cancer probability” in FIG. 12. In addition, the “benign probability” is indicated in four stages of “A” to “D” in descending order. In addition, “tumor size” is indicated in four stages of “A” to “D” in ascending order of “colorectal cancer probability” in FIG. 12. The “presence or absence of metastasis” is indicated in four stages of “A” to “D” in ascending order of the “metastasis probability” in FIG. 12. In a comment field, comments by doctors and screening organizations are described.

There are a wide variety of conditions (cancer type, stage, TNM, malignant/benign, tumor size, activity, angiogenesis, invasion, metastasis, etc.) in a cancer, and it is predicted that there is metabolic reprogramming. Until now, as practical cancer screening, only whether or not it is a cancer (I/O discrimination) has been determined by formula (1). However, in actual cancer screening, it is necessary to present not only such information but also malignancy/benignancy, risk (qualitative probability), therapeutic effect (quantitative variable), and the like. According to the present embodiment, it is possible to generate a cancer screening model capable of determining a probability of onset of a cancer (colorectal cancer), a size of a tumor, and the like, on the basis of intensity of metabolites in a urine specimen. As a result, it is possible to screen various cancer conditions on the basis of the intensity of metabolites in a urine specimen, and it is possible to greatly improve cost and efficiency of cancer screening. In addition, such a cancer screening model may be used as an auxiliary means such as therapeutic assistance such as correspondence with an image diagnosis result, quantitative clarification by being used for interpolation of data, and discovery of an undiscoverable minute tumor by being used for extrapolation of image data.

In addition, in steps S111 and S112, the candidate extraction unit 112 narrows down metabolites to be subjected to generation of the cancer screening model, so that the cancer screening model may be efficiently generated. When narrowing down metabolites, the candidate extraction unit 112 extracts metabolites having a significant difference between cancer patients and healthy subjects by a significance test. Alternatively, the candidate extraction unit 112 ranks metabolites on the basis of an importance level of the random forest and extracts metabolites ranked high. As a result, metabolites related to onset of a cancer may be narrowed down, so that a cancer screening model can be efficiently generated.

In addition, it is possible to determine whether or not a cancer or a predetermined cancer type (for example, colorectal cancer) has developed by the first cancer screening model 142.

Furthermore, the probability of onset of a cancer or a predetermined condition in a predetermined cancer type (for example, colorectal cancer) is estimated by the second cancer screening model 143 and the fourth cancer screening model 145.

Then, a degree of a state of a predetermined phenomenon in a cancer (for example, a size of the tumor, or the like) is estimated by the third cancer screening model 144.

In addition, by generating a cancer screening model as in the present embodiment, the inventor has found that there is a useful marker for each cancer screening model. For example, among the top metabolite candidates for determining whether or not a cancer is present (FIG. 6, and the like), there are metabolites that are suitable for determining high and low of a cancer stage number and metabolites that are not suitable for determining high and low of a cancer stage number.

In the present embodiment, marker candidates are narrowed down by performing a significance test and further performing random forest in step S111 in FIG. 3, but the present invention is not limited thereto. For example, a correlation coefficient between n (for example, n=10) metabolites may be calculated, and candidates for a marker may be narrowed down by excluding those having a high correlation coefficient. Alternatively, there is a method of narrowing down markers by preferentially leaving metabolites in different routes as marker candidates by pathway analysis, or the like.

The present invention is not limited to the above-described embodiment and includes various modifications. For example, the above-described embodiment has been described in detail for explaining the present invention in a lucid way and are not necessarily limited to those having all the described configurations.

In addition, some or all of the above-described configurations, functions, units 111 to 115, DBs 120, 130, 140, 150, 160, and the like, may be implemented by hardware, for example, by being designed with an integrated circuit. In addition, as illustrated in FIG. 2, each of the above-described configurations, functions, and the like, may be implemented by software by a processor such as the CPU 104 interpreting and executing a program for implementing each function. Information such as a program, a table, and a file for implementing each function can be stored in a recording device such as the memory 110 and a solid state drive (SSD) or a recording medium such as an integrated circuit (IC) card, a secure digital (SD) card and a digital versatile disc (DVD) in addition to a hard disk (HD).

In addition, in each embodiment, control lines and information lines considered to be necessary for description are illustrated, and not all control lines and information lines in a product are necessarily illustrated. In practice, it may be considered that almost all the components are connected to each other.

### REFERENCE SIGNS LIST

1 cancer screening device
2 LC/MS
3 user terminal (output unit)
101 communication device (first acquisition unit, second acquisition unit)
103 output device (output unit)
112 candidate extraction unit (narrowing unit)
113 cancer screening model generation unit
114 screening processing unit (cancer state estimation unit)
115 output processing unit (output unit)
121 clinical data (cancer screening data)
131 metabolite exhaustive data (first metabolite exhaustive data)
132 sample data (second metabolite exhaustive data)
142 first cancer screening model (first cancer screening model)
143 second cancer screening model (second cancer screening model)
144 third cancer screening model (third cancer screening model)
145 fourth cancer screening model (second cancer screening model)
S121 first screening model generation processing (cancer screening model generation step)
S122 second screening model generation processing (cancer screening model generation step)
S123 third screening model generation processing (cancer screening model generation step)
S124 fourth screening model generation processing (cancer screening model generation step)
S211 second screening model processing (cancer state estimation step)
S213 third screening model processing (cancer state estimation step)
S214 fourth screening model processing (cancer state estimation step)
S215 fifth screening model processing (cancer state estimation step)
S221 result output (output step)

What is claimed:

1. A cancer screening device comprising:
a first acquisition unit configured to: acquire cancer screening data storing cancer screening results that are results of cancer screening for first subjects who are a plurality of subjects including cancer patients and healthy subjects; and acquire first metabolite exhaustive data that are results of performed LC/MS analyses for first urine specimens collected from the first subjects and is information related to amounts of a plurality of metabolites in the first urine specimens;
a cancer screening model generation unit configured to construct, as a cancer screening model, a relationship between the cancer screening results in the cancer screening data and the respective amounts of the metabolites in the first metabolite exhaustive data on a basis of the cancer screening data and the first metabolite exhaustive data;
a second acquisition unit configured to acquire second metabolite exhaustive data that is a result of a performed LC/MS analysis for a second urine specimen collected from a second subject who is a subject different from the first subjects;
a cancer state estimation unit configured to estimate a state of a cancer in the second subject by applying an amount of metabolite in the second metabolite exhaustive data to the cancer screening model; and
an output unit configured to output the estimated state of the cancer.

2. The cancer screening device according to claim 1, further comprising a narrowing unit configured to narrow down data of the amounts of the metabolites in the first metabolite exhaustive data acquired by the first acquisition unit using a predetermined method.

3. The cancer screening device according to claim 2, wherein the narrowing unit performs a significance test on the amounts of the metabolites in the first metabolite exhaustive data acquired by the first acquisition unit with respect to the cancer screening results to extract the metabolites having a significant difference between cancer patients and healthy subjects.

4. The cancer screening device according to claim 2, wherein
the narrowing unit
calculates importance levels of the metabolites by random forest for the amounts of the metabolites in the first metabolite exhaustive data acquired by the first acquisition unit,
ranks the importance levels, and
extracts metabolites ranked in predetermined numbers from the top.

5. The cancer screening device according to claim 1, wherein the cancer screening model generation unit generates a first cancer screening model for determining whether or not a cancer or a predetermined cancer type has developed on a basis of OPLS-DA.

6. The cancer screening device according to claim 5, wherein the first cancer screening model determines whether or not a colorectal cancer has developed.

7. The cancer screening device according to claim 6, wherein the first cancer screening model is a first-order polynomial having intensity of ion of each of the following metabolites as a variable, intensity of ion being measured by the LC/MS:
(1) metabolites detected as mass spectrum with a mass-to-charge ratio of 91 under a condition where an LC/MS separation mode is reversed phase/negative ionization;
(2) metabolites detected as mass spectrum with a mass-to-charge ratio of 255 under a condition where an LC/MS separation mode is reversed phase/negative ionization;
(3) metabolites detected as mass spectrum with a mass-to-charge ratio of 224 under a condition where an LC/MS separation mode is reversed phase/positive ionization;
(4) metabolites detected as mass spectrum with a mass-to-charge ratio of 168 under a condition where an LC/MS separation mode is reversed phase/negative ionization;
(5) metabolites detected as mass spectrum with a mass-to-charge ratio of 317 under a condition where an LC/MS separation mode is reversed phase/negative ionization;
(6) metabolites detected as mass spectrum with a mass-to-charge ratio of 245 under a condition where an LC/MS separation mode is reversed phase/negative ionization;
(7) metabolites detected as mass spectrum with a mass-to-charge ratio of 288 under a condition where an LC/MS separation mode is reversed phase/positive ionization;
(8) metabolites detected as mass spectrum with a mass-to-charge ratio of 343 under a condition where an LC/MS separation mode is reversed phase/negative ionization;
(9) metabolites detected as mass spectrum with a mass-to-charge ratio of 110 under a condition where an LC/MS separation mode is reversed phase/positive ionization; and
(10) metabolites detected as mass spectrum with a mass-to-charge ratio of 177 under a condition where an LC/MS separation mode is reversed phase/positive ionization.

8. The cancer screening device according to claim 1, wherein the cancer screening model generation unit generates a second cancer screening model for estimating a probability of onset of a predetermined state of a cancer or a predetermined cancer type on a basis of logistic analysis.

9. The cancer screening device according to claim 8, wherein the second cancer screening model estimates a probability of onset of a colorectal cancer.

10. The cancer screening device according to claim 7, wherein the second cancer screening model is a first-order polynomial having intensity of ion of each of the following metabolites as a variable, the intensity of ion being measured by the LC/MS:
(21) metabolites detected as mass spectrum with a mass-to-charge ratio of 91 under a condition where an LC/MS separation mode is reversed phase/negative ionization;
(22) metabolites detected as mass spectrum with a mass-to-charge ratio of 255 under a condition where an LC/MS separation mode is reversed phase/negative ionization;
(23) metabolites detected as mass spectrum with a mass-to-charge ratio of 317 under a condition where an LC/MS separation mode is reversed phase/negative ionization;
(24) metabolites detected as mass spectrum with a mass-to-charge ratio of 288 under a condition where an LC/MS separation mode is reversed phase/positive ionization;
(25) metabolites detected as mass spectrum with a mass-to-charge ratio of 299 under a condition where an LC/MS separation mode is reversed phase/negative ionization;
(26) metabolites detected as mass spectra with a mass-to-charge ratio of 287 under a condition where an LC/MS separation mode is HILIC/negative ionization; and
(27) metabolites detected as mass spectrum with a mass-to-charge ratio of 243 under a condition where an LC/MS separation mode is reversed phase/negative ionization.

11. The cancer screening device according to claim 8, wherein the second cancer screening model is for estimating a probability of malignancy or benignancy of a cancer or a predetermined cancer type on a basis of logistic analysis.

12. The cancer screening device according to claim 1, wherein the cancer screening model generation unit generates a third cancer screening model for determining a degree of a state of a predetermined phenomenon in a cancer on a basis of multiple regression analysis.

13. The cancer screening device according to claim 12, wherein the degree of the state of the predetermined phenomenon is a size of a tumor.

14. The cancer screening device according to claim 13, wherein the third cancer screening model is a first-order polynomial having ionic strength of each of the following metabolites as a variable, the ionic strength being measured by the LC/MS:
(31) metabolites detected as mass spectrum with a mass-to-charge ratio of 91 under a condition where an LC/MS separation mode is reversed phase/negative ionization;
(32) metabolites detected as mass spectrum with a mass-to-charge ratio of 255 under a condition where an LC/MS separation mode is reversed phase/negative ionization;
(33) metabolites detected as mass spectrum with a mass-to-charge ratio of 317 under a condition where an LC/MS separation mode is reversed phase/negative ionization;
(34) metabolites detected as mass spectrum with a mass-to-charge ratio of 343 under a condition where an LC/MS separation mode is reversed phase/negative ionization;
(35) metabolites detected as mass spectrum with a mass-to-charge ratio of 110 under a condition where an LC/MS separation mode is reversed phase/positive ionization; and
(36) metabolites detected as mass spectrum with a mass-to-charge ratio of 177 under a condition where an LC/MS separation mode is reversed phase/positive ionization.

15. A cancer screening method to be executed by a cancer screening device that generates a cancer screening model for estimating a state of a cancer and estimates the state of the cancer in a subject whose state of the cancer is unknown by using the generated cancer screening model, the cancer screening method comprising:
a cancer screening model generation step of generating, as the cancer screening model, a relationship between cancer screening results in cancer screening data and amounts of metabolites in first metabolite exhaustive data on a basis of the cancer screening data storing the cancer screening results that are results of cancer screening for first subjects who are a plurality of subjects including cancer patients and healthy subjects, and the first metabolite exhaustive data that are results of performed LC/MS analyses for first urine specimens collected from the first subjects and is information related to the amounts of a plurality of metabolites in the first urine specimens; and
a cancer state estimation step of estimating a state of a cancer in a second subject by applying an amount of metabolite in second metabolite exhaustive data to the cancer screening model, the amount of the metabolite in the second metabolite exhaustive data being a result of a performed LC/MS analysis for a second urine specimen collected from the second subject who is a subject different from the first subjects; and
an output step of outputting the estimated state of the cancer.
